(19) United States
(12) Patent Application Publication
(10) Pub. No.: US 2021/0275347 A1
(43) Pub. Date: Sep. 9, 2021

(54) INTRAVAGINAL RING DEVICES

(71) Applicant: DARÉ BIOSCIENCE, INC., San Diego, CA (US)

(72) Inventors: David Friend, San Diego, CA (US); Jennifer Kiang, La Jolla, CA (US); Nicolas Pacelli, Cedar Park, TX (US); Mark Walters, San Diego, CA (US)

(21) Appl. No.: 17/331,119

(22) Filed: May 26, 2021

Related U.S. Application Data
(63) Continuation-in-part of application No. PCT/US2020/061058, filed on Nov. 18, 2020.
(60) Provisional application No. 62/937,247, filed on Nov. 18, 2019.

(51) Int. Cl. A61F 6/08; A61K 33/26; C08L 31/04; A61K 33/34; A61K 31/4985; C08L 23/06; C08L 23/12; A61K 47/22

(52) U.S. Cl. CPC A61F 6/08; A61K 33/26; C08L 31/04; A61K 47/22; A61K 31/4985; C08L 23/06; C08L 23/12; A61K 33/34

(57) ABSTRACT

Disclosed herein are intravaginal ring (IVR) devices comprising barriers that may be metal, polymeric or combinations of metal and polymeric. The IVRs are useful for contraception, treating and/or preventing sexually transmitted diseases, and treating and/or preventing bacterial infections. The IVR's are resistant to distortion due to absorption of vaginal fluids. This allows the IVRs to perform better over time as compared to other intravaginal rings made with copolymeric silicone.

### RELATED APPLICATIONS

This patent application is a U.S. bypass continuation-in-part of PCT Application No. PCT/US2020/061058, filed on 18 Nov. 2020, which claims priority to U.S. patent Application No. 62/937,247, filed on 18 Nov. 2019, all of which are incorporated by reference herein in their entireties, including the drawings.

### FIELD OF THE DISCLOSURE

The present disclosure is in the field of women's health. More specifically, the disclosure is directed to intravaginal ring (IVR) devices for use in contraception. The IVR devices are comprised of a non-segmented or segmented ring that is made with an uncoated thermoplastic polymer that encircles a curved or flat non-resorbable barrier, wherein the ring has minimal distortion to its ring structure due to the absorption of vaginal fluid or from humidity that occurs in the storage of the device before its use as compared to intravaginal devices made with a copolymeric silicone matrix.

### BACKGROUND OF THE DISCLOSURE

Intravaginal ring devices made with a copolymeric silicone matrix become distorted due to humidity or when used in a subject for a period of time resulting in an IVR device that may not fit properly in a subject, resulting in an intravaginal device that may not function as well as a non-distorted device. In addition, the incorporation of the spermiostat in the ring matrix potentially weakens the mechanical properties of the ring.

The present disclosure covers new intravaginal ring devices that have ring components and or ring shapes that reduce or eliminate ring distortion over time that occurs with intravaginal ring devices made with a copolymeric silicone matrix enabling a better fit and more comfort for subjects using the device.

### SUMMARY OF THE INVENTION

In an embodiment of the disclosure, disclosed herein are intravaginal ring (IVR) devices that have a flat, circular or oval ring made with a non-segmented or segmented uncoated thermoplastic elastomer, excluding copolymeric silicone. The ring may contain one or more non-hormonal spermiostatic agents, antibacterial, antifungal and/or antiviral agents; and, the ring encircles a curved or flat, non-resorbable metal barrier, polymeric barrier, combination metal and polymeric barrier or a barrier made with perforated film, wherein the barrier is attached to the ring and the barrier is not fully occlusive to allow the passage of uterine secretions.

In another embodiment, disclosed herein are IVR devices comprising a flat, circular or oval ring made with a non-segmented or segmented uncoated thermoplastic elastomer, excluding copolymeric silicone, wherein the ring contains one or more active ingredients; and, wherein the ring encircles a curved or flat, non-resorbable metal barrier, polymeric barrier, combination metal and polymer barrier or a barrier made with perforated film, wherein the barrier is attached to the ring and the barrier is not fully occlusive to allow the passage of uterine secretions.

In another embodiment, the IVR device comprises a flat, circular or oval ring made with a non-segmented or segmented uncoated thermoplastic elastomer, excluding copolymeric silicone, wherein the ring encircles a curved or flat, non-resorbable metal barrier, polymeric barrier, combination metal and polymer barrier or a barrier made with perforated film, wherein the barrier is attached to the ring and said metal and polymer barriers and said perforated film contain active ingredients; wherein said barriers or perforated film is not fully occlusive to allow the passage of uterine secretions.

In some embodiments, the IVR comprises a flat, circular or oval ring made with a non-segmented uncoated thermoplastic elastomer, excluding copolymeric silicone, wherein the ring contains one or more non-hormonal spermiostatic agents; and, wherein the ring encircles a curved, non-resorbable polymeric barrier which is attached to the ring; and wherein the ring covers the cervix when inserted into a subject. In further embodiments of this IVR device, the flat, circular or oval intravaginal ring is made with a segmented uncoated thermoplastic elastomer. In some embodiments of the flat, circular or oval intravaginal ring, the number of segmented ring sections is at least two.

In some embodiments, the uncoated thermoplastic elastomer of the flat, circular or oval intravaginal ring is selected from one or more of styrene-butadiene block copolymer, ethylene vinyl acetate copolymer, poly(methyl methacrylate), poly(butyl methacrylate), poly(vinyl chloride), nylon, soft nylon, poly(ethylene terephthalate) (PET), poly(ethylene), poly(acrylonitrile), polychlorotrifluoroethylene (PCTFE), poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

In some embodiments, the uncoated thermoplastic elastomer for the flat, circular or oval intravaginal ring is selected from one or more of ethylene vinyl acetate copolymer (EVA), polyurethane, or polyethylene terephthalate (PET).

In some embodiments, the uncoated thermoplastic elastomer for the flat, circular or oval intravaginal ring is EVA.

In some embodiments, the flat, circular or oval IVR device has a polymeric barrier that is a mesh that is comprised of one or more of metal, polyolefin, nylon, and/or silk. In some embodiments, the polyolefin is polypropylene or polyethylene. In other embodiments, the polymeric mesh of the flat, circular or oval IVR device is a mono- or multi-filament polymer. In some embodiments of the flat, circular or oval IVR device, the polymeric barrier or mesh comprises one or more spermiostatic metals and/or metal salts as active ingredients. In some embodiments, the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

In some embodiments of the flat, circular or oval IVR device, the IVR device has a metal barrier comprising one or more of aluminum, copper, stainless steel, titanium, nickel, nickel-titanium, and precious metals, such as, without limitation gold, silver, platinum or palladium. In some embodiments, when the metal barrier is comprised of copper or silver, the metal barrier will have spermiostatic effects. In some embodiments, the metal barrier is a mono- or multi-filament polymer.

In some embodiments, the flat, circular or oval IVR device contains two or more segments.

In another embodiment, the flat, circular or oval IVR devices have barriers that are a combination of metals and polymers, wherein the combination barrier is attached to the ring. In some embodiments of this aspect, the metal and/or polymer can be a mono or multifilament. In some embodiments, the polymeric barrier portion is comprised of one or more polyolefin, nylon, and/or silk. In other embodiments, the polyolefin is polypropylene or polyethylene. In some embodiments, the metal portion of the barrier comprises one or more of aluminum, copper, stainless steel, titanium, nickel, nickel-titanium, gold, silver, platinum or palladium. In some embodiments, the metal and/or polymeric portion of the barrier contains one or more spermiostatic metals and/or metal salts as active ingredients. In some embodiments, the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

In some embodiments of the flat, circular or oval IVR devices, the use of metal salts results in an intravaginal ion concentration range of about 0.5 to about 20 μM per day. In some embodiments, the spermiostatic metals and/or metal salts are part of the ring. In other embodiments, the metals and or metal salts are part of the barrier or mesh. In some embodiments metal ion release may act as both a spermiostat and an anti-bacterial agent.

Also disclosed herein, are flat, circular or oval IVR devices wherein the barrier is a perforated film. In some embodiments, the perforated film comprises multiple layers that are offset. In some embodiments, the perforated film is made with polymers such as 1,2-polybutadiene, ethylene vinyl acetate, polyethylene, silicone gel, or polyurethane.

In some embodiments, the flat, circular or oval IVR devices disclosed herein also contain ascorbic acid. In some embodiments, the ascorbic acid is part of the ring. In other embodiments, the ascorbic acid is part of the barrier such as the metal barrier, polymeric barrier, combination metal and polymeric barrier or perforated film barrier.

In some embodiments, the flat, circular or oval IVR devices disclosed herein, the polymeric barrier, metal barrier, combination metal/polymeric barrier, or perforated film barrier have pores that range in size from about 100-150 μm 80 μm to about 150 μm or from about 80 μm to about 130 μm, or about 80 μm to about 90 μm, or about 80 μm to about 100 μm, or about 80 μm to about 110 μm, or about 80 μm to about 120 μm.

As used herein term “about” is used to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

In some embodiments, the flat, circular or oval IVR device has a removal tab, string or tag or similar structure that aids in removing the ring after insertion. In some embodiments the removal tab has a diameter of about 10 mm to about 20 mm in length and is integrated into the outer ring structure. In some embodiments, the tab is part of the ring and the tag is part of the barrier. In some embodiments, the tab is made from the same material as the ring and the tag is made from the same material as the barrier. In some embodiments, the string is integrated into the barrier and extends distally about 30 mm from the intravaginal ring.

In some embodiments, the flat, circular or oval ring has an active agent to prevent pregnancy, and/or treat or prevent bacterial infections, fungal infections, and/or viral infections. In some embodiments, the active agent is a non-hormonal or hormonal contraceptive, and the antiviral agent is used to treat or prevent one or more sexually transmitted infections. In some embodiments, the antiviral agent is one or more of tenofovir, atanzanavir, darunavir, fos/amprenavir, indinavir, lopinavir, nelfinavir, ritonavir, saquinavir, tipranavir, efavirenz, or nelfinavir. In some embodiments, the hormonal contraceptive is one or more of desogestrel, drospirenone, ethinyl estradiol, levonorgestrel, medroxyprogesterone acetate, norelgestromin, norethindrone, norgestimate, or norgestrel. In some embodiments, the intravaginal ring comprises active ingredients contained in discrete reservoirs and/or continuous sheathed in a polymer as part of the thermoplastic elastomer as shown in FIG. 7.

In another aspect of the disclosure, the flat, circular or oval ring device is resistant to shape distortion due to absorption of vaginal fluids and/or fluids contained in the packaging used to store the device before use.

In some embodiments, the flat, circular or oval ring IVR device absorbs less water or simulated vaginal fluid as compared to an intravaginal ring made with a copolymeric silicone matrix in which both rings contain equivalent amounts and composition of nonhormonal spermiostatic agents and or ascorbic acid.

In some embodiments, the amount of absorption of simulated vagina fluid by the flat circular ring intravaginal device is about 95% less, 90% less, 85% less, 80% less, 70% less, 60% less, 50% less, 40% less, 30%, 20% less, 10%, less, 5% less, 4% less, 3% less, 2% less or 1% less as compared to an intravaginal ring made with a copolymeric silicone matrix in which both rings contain equivalent amounts and composition of nonhormonal spermiostatic agents.

In some embodiments, the amount of simulated vaginal fluid absorption of the flat, circular ring intravaginal device is from about 1%-95% less, or any ranges in between about 1%-95% less as compared to an intravaginal ring made with a copolymeric silicone matrix in which both rings contain equivalent amounts and composition of nonhormonal spermiostatic agents.

In some embodiments of the disclosure, disclosed herein are IVR devices comprising a saddle-shaped, oval ring made with a non-segmented or segmented uncoated thermoplastic elastomer, excluding copolymeric silicone, wherein the ring contains one or more active ingredients; and, wherein the ring encircles a curved, non-resorbable metal barrier, polymeric barrier, combination metal and polymer barrier or a barrier made with perforated film, wherein the barrier is attached to the ring and the barrier is not fully occlusive to allow the passage of uterine secretions.

In another embodiment, disclosed herein are intravaginal ring devices comprising a saddle-shaped, oval ring made with a non-segmented or segmented uncoated thermoplastic elastomer, excluding copolymeric silicone, wherein the ring encircles a curved or flat, non-resorbable metal barrier, polymeric barrier, combination metal and polymer barrier or a barrier made with perforated film, wherein the barrier is attached to the ring and said metal and polymer barriers and said perforated film optionally contain active ingredients; wherein said barriers or perforated film is not fully occlusive to allow the passage of uterine secretions.

In another embodiment, disclosed herein are IVR devices comprising a saddle-shaped, oval ring made with a non-segmented uncoated thermoplastic elastomer, excluding copolymeric silicone, wherein the ring contains one or more non-hormonal spermiostatic ingredients; and, wherein the ring encircles a curved, non-resorbable polymeric barrier which is attached to the ring; and wherein the ring covers the cervix.

In another embodiment of the disclosure, disclosed herein are intravaginal ring devices that are a saddle-shaped, oval ring made with a non-segmented or segmented uncoated thermoplastic elastomer, excluding copolymeric silicone. The saddle-shaped intravaginal ring contains one or more non-hormonal spermiostatic agents or hormonal contraceptives; and, the saddle-shaped oval ring encircles a curved, non-resorbable metal barrier, polymeric barrier, combination metal and polymer barrier or a barrier made with perforated film, wherein the barrier is attached to the ring and the barrier is not fully occlusive to allow the passage of uterine secretion.

In some embodiments, the thermoplastic elastomer of the saddle-shaped, oval ring is selected from the group of one or more of styrene-butadiene block copolymer, ethylene vinyl acetate copolymer, poly(methyl methacrylate), poly(butyl methacrylate), poly(vinyl chloride), nylon, soft nylon, poly(ethylene terephthalate) (PET), poly(ethylene), poly(acrylonitrile), polychlorotrifluoroethylene (PCTFE), poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

In some embodiments, the uncoated thermoplastic elastomer for the saddle-shaped, oval ring IVR device is selected from one or more of ethylene vinyl acetate copolymer, polyurethane, or PET.

In some embodiments, the saddle-shaped, oval ring IVR device has a polymeric barrier mesh that is comprised of one or more of polyolefin, nylon, and/or silk. In some embodiments, the polyolefin is polypropylene or polyethylene. In some embodiments, the polymeric barrier mesh is a mono- or multi-filament polymer. In some embodiments of the saddle-shaped, oval ring IVR device, the polymeric barrier or mesh comprises one or more spermiostatic metals and/or metal salts as active ingredients. In some embodiments, the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

In some embodiments, the use of metal salts results in an intravaginal ion concentration range of about 0.5 to about 20 μM per day. In some embodiments, the spermiostatic metals and/or metal salts are part of the ring. In other embodiments, the metals and or metal salts are part of the barrier or mesh. In some embodiments metal ion release may act as both a spermiostat and an anti-bacterial agent.

In some embodiments the saddle-shaped, oval ring intravaginal device has a metal barrier comprising one or more of aluminum, copper, stainless steel, titanium, nickel, nickel-titanium, and precious metals, such as, without limitation gold, silver, platinum or palladium. In some embodiments, when the metal barrier is comprised of copper or silver, the metal barrier will have spermiostatic effects. In some embodiments, the metal barrier is a mono- or multi-filament polymer.

In some embodiments the saddle-shaped, oval ring intravaginal device contains two or more segments.

In another embodiment, the saddle-shaped, oval ring intravaginal devices disclosed herein have barriers that are a combination of metals and polymers, wherein the combination barrier is attached to the ring. In some embodiments, the metal and/or polymer can be a mono or multifilament. In some embodiments, the polymeric barrier portion is comprised of one or more polyolefin, nylon, and/or silk. In other embodiments, the polyolefin is polypropylene or polyethylene. In some embodiments, the metal portion of the barrier comprises one or more of aluminum, copper, stainless steel, titanium, nickel, nickel-titanium, gold, silver, platinum or palladium. In some embodiments, the metal and/or polymeric portion of the barrier contains one or more spermiostatic metals and/or metal salts as active ingredients. In some embodiments, the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

Also disclosed herein are saddle-shaped, oval ring IVR devices wherein the barrier is a perforated film. In some embodiments, the perforated film comprises multiple layers that are offset. In some embodiments, the perforated film is made with polymers such as 1,2-polybutadiene, ethylene vinyl acetate, polyethylene, silicone gel, or polyurethane.

In some embodiments, the saddle-shaped, oval ring IVR devices disclosed herein also contain ascorbic acid. In some embodiments, the ascorbic acid is part of the ring. In other embodiments, the ascorbic acid is part of the barrier such as the metal barrier, polymeric barrier, combination metal and polymeric barrier or perforated film barrier.

In some embodiments, the saddle-shaped, oval ring IVR devices disclosed herein, the polymeric, metal, combination metal/polymeric, or perforated film barrier have pores that range in size from about 100-150 μm 80 μm to about 150 μm or from about 80 μm to about 130 μm, or about 80 μm to about 90 μm, or about 80 μm to about 100 μm, or about 80 μm to about 110 μm, or about 80 μm to about 120 μm.

In some embodiments, the saddle-shaped, oval ring IVR devices have a removal tab, string or tag or similar structure that aids in removing the ring after insertion. In some embodiments the removal tab has a diameter of about 10 mm to about 20 mm in length and is integrated into the outer ring structure. In some embodiments, the tab is part of the ring and the tag is part of the barrier. In some embodiments, the tab is made from the same material as the ring and the tag is made from the same material as the barrier. In some embodiments, the string is integrated into the barrier and extends distally about 30 mm from the intravaginal ring.

In some embodiments, the saddle-shaped, oval ring IVR devices have an active agent to prevent pregnancy, and/or treat or prevent bacterial infections, fungal infections, and/or viral infections. In some embodiments, the active agent is a non-hormonal or hormonal contraceptive, and the antiviral agent is used to treat or prevent one or more sexually transmitted infections. In some embodiments, the antiviral agent is one or more of tenofovir, atanzanavir, darunavir, fos/amprenavir, indinavir, lopinavir, nelfinavir, ritonavir, saquinavir, tipranavir, efavirenz, or nelfinavir. In some embodiments, the hormonal contraceptive is one or more of desogestrel, drospirenone, ethinyl estradiol, levonorgestrel, medroxyprogesterone acetate, norelgestromin, norethindrone, norgestimate, or norgestrel. In some embodiments, the intravaginal ring comprises active ingredients contained in discrete reservoirs and/or continuous sheathed in a polymer as part of the thermoplastic elastomer as shown in FIG. 7.

In some embodiments, the saddle-shaped, oval ring intravaginal device absorbs less simulated vaginal fluid or water as compared to an intravaginal ring made with a copolymeric silicone matrix in which both rings contain equivalent amounts and composition of nonhormonal spermiostatic agents and ascorbic acid if present.

In some embodiments, the amount of simulated vaginal fluid absorption of the saddle-shaped, oval ring intravaginal device is about 95% less, 90% less, 85% less, 80% less, 70% less, 60% less, 50% less, 40% less, 30%, 20% less, 10%, less, 5% less, 4% less, 3% less, 2% less or 1% less as compared to an intravaginal ring made with a copolymeric silicone matrix in which both rings contain equivalent amounts and composition of nonhormonal spermiostatic agents.

In some embodiments, the amount of water absorption of the saddle-shaped, oval ring intravaginal device is from about 1%-95%, or any ranges in between about 1%-95% less as compared to an intravaginal ring made with a copolymeric silicone matrix in which both rings contain equivalent amounts and composition of nonhormonal spermiostatic agents.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The Figures illustrate exemplary modes of aspects and embodiments of the disclosure. However, the scope of the invention is not limited to the specific embodiments disclosed in these Figures, which are for purposes of illustration only, since alternative embodiments can be utilized to obtain similar results.

FIG. 1 is a perspective view of an intravaginal ring device of the disclosure with a curved polymeric barrier, and flat circular ring.

FIG. 2 is a side view of the intravaginal device of FIG. 1 also showing the rounded or curved cross section of the ring.

FIG. 3 is a perspective view of an intravaginal ring device of the disclosure with a saddle-shaped ring and curved polymeric barrier.

FIG. 4 is a side view of the intravaginal ring device of FIG. 3 also showing the rounded or curved cross section of the saddle-shaped ring.

FIG. 5 is a perspective view of an intravaginal ring device of the disclosure with a segmented ring, curved polymeric barrier and removal tap.

FIG. 6 is a side view of the intravaginal device of FIG. 5 also showing the rounded or curved cross section of the ring.

FIG. 7 is a plan and cross-sectional view of the ring portion of the intravaginal device with drug containing reservoirs formed into the ring body in various configurations.

### DETAILED DESCRIPTION OF THE EMBODIMENTS

Disclosed herein are intravaginal ring (IVR) devices. In some embodiments, the intravaginal ring devices include a flat, circular or oval ring made with a non-segmented or segmented, uncoated, thermoplastic elastomer, excluding copolymeric silicone. The ring encircles a curved, non-resorbable polymeric barrier, metal barrier, combination metal/polymeric barrier or perforated film barrier which are attached to the ring; and wherein the ring covers the cervix.

In other embodiments, the IVR devices disclosed herein are a saddle-shaped oval ring made with a non-segmented or segmented uncoated thermoplastic elastomer. The ring encircles a curved, non-resorbable polymeric barrier, metal barrier, combination metal/polymeric barrier or perforated film barrier which is attached to the ring; and wherein the ring covers the cervix.

In some embodiments, the IVR devices disclosed herein have barriers that are not fully occlusive to allow the passage of uterine secretions.

As used herein thermoplastic elastomers are copolymers or a mix of polymers that have thermoplastic and elastomeric properties and exclude silicone polymers.

Thermoplastic polymers suitable for IVR devices disclosed herein include polymers and copolymers that are capable of being softened by heating and hardened by cooling through a temperature range characteristic of the polymer, its crystalline melting or glass transition temperature, and in the softened state they can be shaped by flow into systems by molding or extrusion. Thermoplastic polymers suitable for the present purpose are permeable to non-hormonal spermiostatic agents, ascorbic acid, antimicrobials, antifungals, and absorb a low amount of vaginal fluid when inserted into a subject.

Examples of thermoplastic polymers that can be used to make the disclosed IVR devices include, without limitation, ethylene vinyl acetate copolymer, poly(methyl methacrylate), poly(butyl methacrylate), poly(vinylchloride), nylon, soft nylon, poly(ethylene terephthalate) (PET), poly(ethylene), PCTFE (polychlorotrifluorethylene), poly(ethylene-vinyl esters), poly(ethylene-vinyl acetate), poly(vinylchloride-diethyl fumarate), poly(esters of acrylic and methacrylic), poly(amides), poly(vinyl chloride), poly(urethane), polypropylene, and other poly(olefins). These polymers and their physical properties are known to the art and they can be synthesized according to the procedures disclosed in Encyclopedia of Polymer Science and Technology, Vol. 15, pages 508 to 530, 1971, published by Interscience Publishers, Inc., New York; Polymers, Vol. 17, 938 to 956, 1976; Technical Bulletin SCR-159, 1965, Shell Corp., New York; and references cited therein.

In some embodiments, the rings of the disclosed IVR devices contain one or more spermiostatic metals and/or metal salts. The salts can be selected from one or more of calcium chloride, magnesium chloride, ferrous sulfate, ferrous gluconate, iron amino acid chelates, copper sulfate, copper gluconate, silver nitrite, copper amino acid chelates, and copper oxide.

In some embodiments, the barrier of the intravaginal devices disclosed herein is a mesh comprised of one or more of metal, polyolefin, nylon, and/or silk. In some embodiments, the metal has spermiostatic effects and/or antibacterial effects by incorporation of filaments into the mesh/barrier such as copper and silver. Other metals such as aluminum, stainless steel, titanium, nickel, nickel-titanium, and precious metals, such as, without limitation gold, platinum and palladium are useful for mechanical strength of the barrier. In some embodiments, the metal mesh also contains one or more metal salts, such as iron salts to provide iron ions for spermiostatic effect. In some embodiments, the polyolefin is polypropylene or polyethylene. In some embodiments, the polymeric barrier, or metal barrier is a mono- or multi-filament. In some embodiments the polymeric barrier mesh and/or metal barrier mesh has pores that range in size from about 100-150 μm. In some embodiments the polymeric or metal barrier mesh has pores that range in size from about 80 μm to about 150 μm or from about 80 μm to about 130 μm, or about 80 μm to about 90 μm, or about 80 μm to about 100 μm, or about 80 μm to about 110 μm, or about 80 μm to about 120 μm.

In other embodiments the barrier is made with a perforated film that can be made of multiple layers in which the perforated layers are offset. In some embodiments the film can be made with such polymers as, without limitation, 1,2-polybutadiene, ethylene vinyl acetate, polyethylene, silicone gel, and polyurethane. In some embodiments, metal filaments and/or metal salts are incorporated into at least the outermost perforated layers. In some embodiments the perforated film has pores that range in size from about 80 μm to about 150 μm or from about 80 μm to about 130 μm, or about 80 μm to about 90 μm, or about 80 μm to about 100 μm, or about 80 μm to about 110 μm, or about 80 μm to about 120 μm.

The pore size for the polymer mesh, metal mesh or perforated film can be measured microscopically.

In an embodiment of the disclosed IVR devices, the intravaginal rings, can use multi-purpose prevention technology (MPT) which combines protection against unintended pregnancy, HIV and other sexually transmitted infections. In some embodiments the intravaginal ring can contain one or more segments with each segment containing a separate active ingredient, such as an antiviral agent, e.g., tenofovir, dapivirine, atanzanavir, darunavir, fos/amprenavir, indinavir, lopinavir, nelfinavir, ritonavir, saquinavir, tipranavir, efavirenz, cabotegravir or nelfinavir; or non-hormonal contraceptives, such metal salts as described herein; or hormonal contraceptives, such as segesterone, desogestrel, drospirenone, ethinyl estradiol, levonorgestrel, medroxyprogesterone acetate, norelgestromin, norethindrone, norgestimate, or norgestrel. In some embodiments, the concentration of antiviral agents results in a dose between about 0.1 and about 10 mg per day. In some embodiments, the concentration of hormonal agents results in a dose between about 0.01 and about 2 mg per day.

In some embodiments of the MPT, the non-hormonal is contained in or on the barrier mesh, such as by incorporation into the mesh material or by spraying the non-hormonal onto the barrier mesh. In another embodiment, the barrier mesh contains a low dose of a hormonal contraceptive instead of a non-hormonal metal salt. In another embodiment, the barrier mesh contains both a non-hormonal contraceptive and low dose hormonal contraceptive.

In some embodiments of the IVRs disclosed herein, the active pharmaceutical ingredient(s) (API) can be deposited into a preformed reservoir in the polymer ring and optionally covered and sealed with an additional polymer structure using techniques known in the art such as, without limitation, molding, adhesion, welding etc.)

In some embodiments, the antivirals, antimicrobials and/or antifungal agents are present in the ring portion of the IVR devices. In other embodiments, the antivirals, antimicrobials and/or antifungal agents are present only in the barrier of the IVR devices. In some embodiments, the antivirals, antimicrobials and/or antifungal agents are found in the barrier and/or ring of the IVR devices.

In some embodiments, the IVR devices do not absorb as much vaginal fluid from the subject using the device as compared to a intravaginal ring devices made from a copolymeric silicone matrix.

The ability of the IVR devices of the present disclosure to absorb less vaginal fluid or water as compared to vaginal rings made of polymeric silicone can be tested in vitro using fluids such as water or simulated vaginal fluid. Example 6 provides a method for testing the absorption of simulated vaginal fluid by IVR devices.

In some embodiments, the IVR devices disclosed herein have less distortion than intravaginal ring devices made from a copolymeric silicone matrix when used in a subject for a period of time of about 14-days to about three-months or when stored in packaging that is susceptible to humidity before use. Thus, the disclosed rings will maintain their shape longer than intravaginal ring devices made from a copolymeric silicone matrix, resulting in a better fit, performance and comfort in subjects using the disclosed devices.

In some embodiments, the IVR contain ascorbic acid.

In some embodiments, the IVR devices can be used for greater than at least 14 days.

In some embodiments, the intravaginal ring devices disclosed herein are non-resorbable in the subject.

In some embodiments, the devices disclosed herein, can be used for about 30 to 31 days, or about 1 month to about 3 months. In some embodiments, the devices disclosed herein can be removed during the menstrual cycle, cleaned, and reused after multiple menstrual cycles.

Example 4 describes one embodiment of producing an IVR device as disclosed herein that has a spermiostatic barrier. Example 4 also describes the preparation of a barrier mesh using a 3D-knitting machine.

Example 5, describes one embodiment of preparing a IVR device in which the ring contains an active agent and the barrier mesh contains a spermiostatic barrier.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, because alternative methods can be utilized to obtain similar results.

### EXAMPLES

### Example 1: Flat Ring

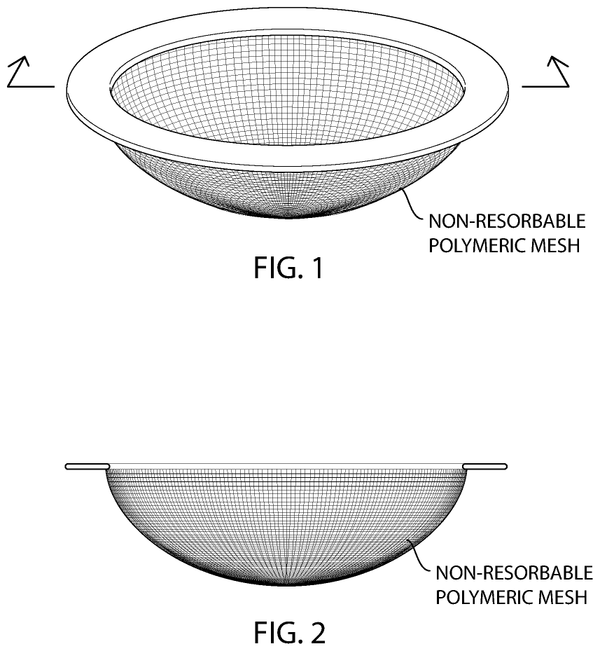

The barrier mesh of the ring device illustrated in FIG. 1 is prepared by extruding poly(propylene), nylon, or poly(ethylene), multi- or monofilaments and using a 3D knitting machine and a warp knit pattern to create a spherical cap shape. The spherical cap shape has an outer dimeter of about 50 mm (millimeters) and a height of about 15 mm.

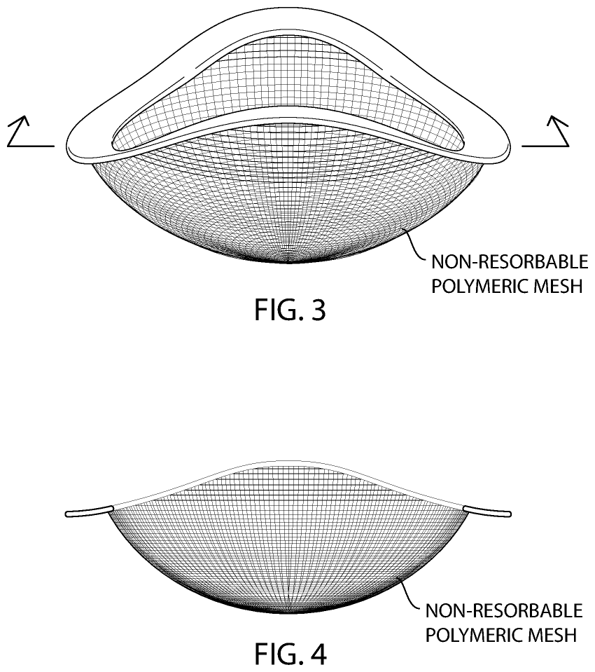

Alternatively, sericin-free silk fibroin multi- or monofilaments are knitted using a 3D knitting machine and a warp knit pattern to create a spherical cap-type shape where the portion of the sphere is cut off by a saddle, instead of a plane (FIG. 3). The spherical cap shape has an outer dimeter of about 50 mm (millimeters) and a height of about 15 mm.

The intravaginal contraceptive ring device illustrated in FIG. 1 is prepared using poly(ethyl-co-vinyl acetate) (EVA), about 500 (milligrams) mg of ferrous gluconate and about 400 mg of ascorbic acid which are dissolved together in approximately 10 mL of a nonpolar solvent such as dichloromethane in a scintillation vial. Next, the polymeric mixture is prepared by adding about 4000 mg of EVA to the solution, and mixing the EVA/drug compositions using a rotary shaker. The resulting mixtures are then solvent cast in dry ice using ethanol as the solvent. The solvent is allowed to evaporate overnight, and the dry EVA/drug mixtures are then ground into powders. The EVA/drug powders are placed into an injection molding unit. The injector is heated to approximately 80° C. The barrier mesh is held in a stainless-steel mold using an insert molding fixture, then the molten EVA/drug compositions are extruded into a stainless-steel mold, creating a finished device with an outer diameter of about 55 mm and a cross-section of about 4 mm.

Alternatively, the intravaginal contraceptive ring device illustrated in FIG. 1 is prepared using polyurethane or polyethylene terephthalate as the ring material and copper gluconate and ascorbic acid as the spermiostatic agents using a process similar to that described above.

### Example 2: Saddle Ring

The barrier mesh of the ring device illustrated in FIG. 3 is prepared by extruding poly(propylene), nylon, or poly(ethylene), multi- or monofilaments and using a 3D knitting machine and a warp knit pattern to create a spherical cap-type shape where the portion of the sphere is cut off by a saddle, instead of a plane (FIG. 3). The spherical cap shape has an outer dimeter of about 50 mm (millimeters) and a height of about 15 mm.

Alternatively, sericin-free silk fibroin multi- or monofilaments are knitted using a 3D knitting machine and a warp knit pattern to create a spherical cap-type shape where the portion of the sphere is cut off by a saddle, instead of a plane (FIG. 3). The spherical cap shape has an outer dimeter of about 50 mm (millimeters) and a height of about 15 mm.

### Example 3: Flat Ring with Removal Tab

The barrier mesh of the ring device is prepared using the process from Example 1 above.

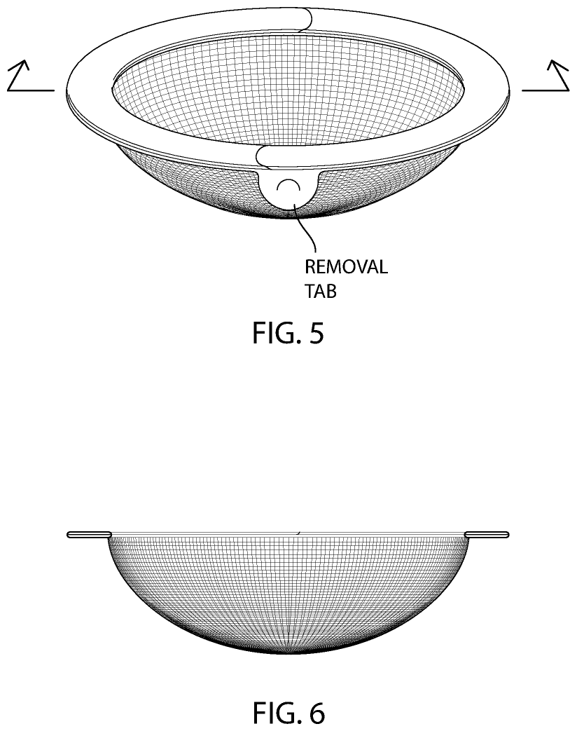

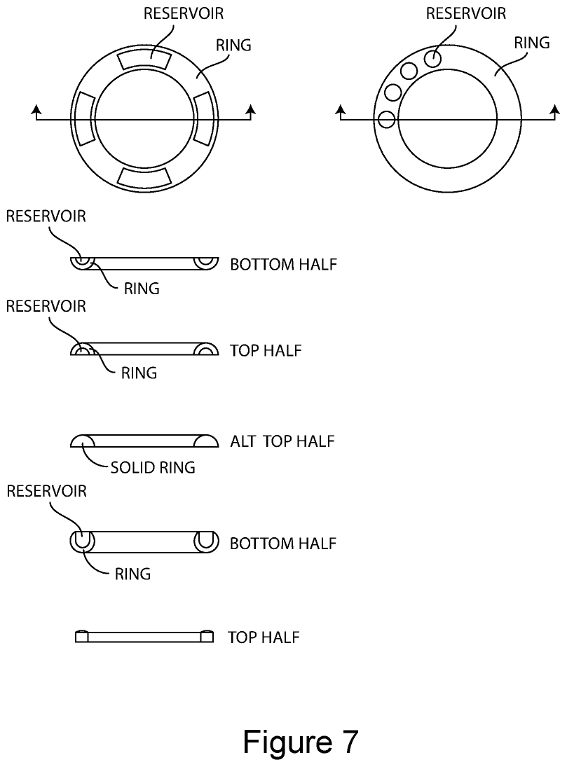

The intravaginal contraceptive ring device illustrated in FIG. 5 is prepared using poly(ethyl-co-vinyl acetate) (EVA). About 250 (milligrams) mg of ferrous gluconate are dissolved in approximately about 5 mL of a nonpolar solvent such as dichloromethane in a scintillation vial. Next, the polymeric mixture is prepared by adding about 2000 mg of EVA to the solution and mixing the EVA/drug composition using a rotary shaker.

A semi-circle of EVA/ascorbic acid is prepared using the process described above. The two semicircles are then welded together into a full circle with an outer diameter of about 55 mm and a semi-circular cross-sectional geometry.

EVA is then placed into an injection molding unit. The mold is circular with an outer diameter of about 55 mm with a removal tab protruding from one side of the circle, and has a semi-circular cross-sectional diameter of about 4 mm. The injector is heated to approximately 80° C., then the molten EVA/ferrous gluconate is extruded into a stainless-steel mold. The two ring halves are then welded together with the barrier mesh in the middle to create the finished device.

Alternatively, the intravaginal contraceptive ring device illustrated in FIG. 5 is prepared using polyurethane, or polyethylene terephthalate as the ring material and copper gluconate and ascorbic acid as the spermiostatic agents using a process similar to that described above.

### Example 4—IVR Ring Device with Spermiostatic Barrier

The intravaginal ring device is produced using EVA polymer and an injection molding method similar to Example 1; however, no drug or spermiostatic agent is incorporated into the polymer mixture prior to molding.

The barrier mesh of the ring device is prepared by extruding poly(propylene), nylon, or poly(ethylene), multi- or monofilaments as well as copper or silver multi- or monofilaments and using a 3D knitting machine and a warp knit pattern to create a spherical cap shape integrating the polymer and metal filaments into a single integrated structure. The spherical cap shape has an outer dimeter of about 50 mm (millimeters) and a height of about 15 mm.

Alternatively, the barrier mesh of the ring device is prepared by combining metal salts (e.g. copper gluconate) and suitable polymers in a method similar to that described in Example 1 and extruding multi- or monofilaments of composite metal salt/polymer filaments and using a 3D knitting machine and a warp knit pattern to create a spherical cap shape containing the spermiostatic agent.

### Example 5—IVR Ring Device with Active Agent and Spermiostatic Barrier

The intravaginal contraceptive ring device illustrated in FIG. 1 is prepared by blending about 2200 g of poly(ethyl-co-vinyl acetate) (EVA) and 800 g of an active agent (e.g. cabotegravir) both in dry powder form. The polymer/drug powder premix is then placed in a screw extruder to produce drug-loaded polymer pellets. The extruder is operated at a haul speed of about 4 meters per minute, a melt temperature of about 85° C., and a melt pressure of about 35 bar. The EVA/drug pellets are placed into an injection molding unit. The injector is heated to approximately 80° C. The barrier mesh is held in a stainless-steel mold using an insert molding fixture, then the molten EVA/drug compositions are extruded into a stainless-steel mold, creating a finished device with an outer diameter of about 55 mm and a cross-section of about 4 mm.

The barrier mesh of the ring device is prepared by extruding poly(propylene), nylon, or poly(ethylene), multi- or monofilaments as well as copper or silver multi- or monofilaments and using a 3D knitting machine and a warp knit pattern to create a spherical cap shape integrating the polymer and metal filaments into a single integrated structure. The spherical cap shape has an outer dimeter of about 50 mm (millimeters) and a height of about 15 mm.

Alternatively, the barrier mesh of the ring device is prepared by combining metal salts (e.g. copper gluconate) and suitable polymers in a method similar to that described in Example 1 and extruding multi- or monofilaments of composite metal salt/polymer filaments and using a 3D knitting machine and a warp knit pattern to create a spherical cap shape containing the spermiostatic agent.

### Example 6—Measurement of Absorption of Synthetic Vaginal Fluid

The IVR device of the present disclosure is weighed with an analytical balance. It is then submerged in about 100 mL of simulated vaginal fluid (SVF) for 14-35 days at 37° C.—for making simulated vaginal fluid see, for example, Rastogi R. et al., (2016), “Engineering and Characterization of Simplified Vaginal and Seminal Fluid Simulants”, Contraception, 2016, 93(4):337-346. The ring is then removed from the simulated vaginal fluid, patted down with a Kimwipe, then weighed again. The ring is then placed in a vacuum oven to remove any water absorbed. The ring is kept in the vacuum oven and dried until it is at a constant weight. That final dry weight is recorded.

The same process is performed with the comparator ring. The water absorption is quantified by subtracting the wet ring weight from the dry ring weight to get the weight of water absorbed per ring. The weight of water absorbed by the comparator ring is divided by the weight of water absorbed by the comparator ring to quantify the percentage less water absorption.

The examples and embodiments described herein are for illustrative purposes only and various modifications or changes suggested to persons skilled in the art are to be included within the spirit and purview of this application and scope of the appended claims.

What is claimed is:

1. An intravaginal ring device comprising a flat, circular or oval ring made with a non-segmented or segmented uncoated thermoplastic elastomer, excluding copolymeric silicone, wherein the ring contains one or more active ingredients; and, wherein the ring encircles a curved or flat, non-resorbable metal barrier, polymeric barrier, combination metal and polymer barrier or a barrier made with perforated film, wherein the barrier is attached to the ring and the barrier is not fully occlusive to allow the passage of uterine secretions.

2. The intravaginal ring device according to claim 1, wherein the uncoated thermoplastic elastomer is selected from one or more of styrene-butadiene block copolymer, ethylene vinyl acetate copolymer, poly(methyl methacrylate), poly(butyl methacrylate), poly(vinylchloride), nylon, plasticized soft nylon, poly(ethylene terephthalate) (PET), poly(ethylene), poly(acrylonitrile), polychlorotrifluoroethylene (PCTFE), poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

3. The intravaginal device according to claim 2, wherein the uncoated thermoplastic elastomer is selected from ethylene vinyl acetate copolymer, polyurethane, or PET.

4. The intravaginal ring device according to claim 3, wherein the uncoated thermoplastic elastomer is ethylene vinyl acetate copolymer.

5. The intravaginal ring device according to any one of claims 1-4, wherein the polymeric barrier is a mesh comprised of one or more of polyolefin, nylon, and/or silk.

6. The intravaginal ring device of claim 5, wherein the polyolefin is polypropylene or polyethylene.

7. The intravaginal ring device according to any one of claims 1-6 wherein the polymeric mesh is a mono- or multi-filament polymer.

8. The intravaginal ring device according to claim 7, wherein the polymeric barrier or mesh comprises one or more spermiostatic metals and/or metal salts as active ingredients.

9. The intravaginal ring device according to claim 8, wherein the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

10. The intravaginal ring device according to any one of claims 1-4, wherein the barrier is metal and comprises one or more of aluminum, copper, stainless steel, titanium, nickel, nickel-titanium, gold, silver, platinum or palladium.

11. The intravaginal ring device according to 10, wherein the metal barrier is a mono- and/or multi-filament metal barrier.

12. The intravaginal ring device according to claim 11, wherein the metal barrier comprises one or more spermiostatic metals.

13. The intravaginal ring device according to claim 12, wherein the metal is copper and/or silver.

14. The intravaginal ring device according to any one of claims 1-4, wherein the barrier is a combination of metal and a polymer barrier which is attached to the ring.

15. The intravaginal ring device according to claim 14, wherein the polymeric barrier is a mesh comprised of one or more of polyolefin, nylon, and/or silk.

16. The intravaginal ring device of claim 15, wherein the polyolefin is polypropylene or polyethylene.

17. The intravaginal ring device according to any one of claim 15 or 16 wherein the polymeric mesh is a mono- or multi-filament polymer.

18. The intravaginal ring device according to any one of claims 14-17, wherein the metal portion of the barrier comprises one or more of aluminum, copper, stainless steel, titanium, nickel, nickel-titanium, gold, silver, platinum or palladium.

19. The intravaginal ring device according to 18, wherein the metal barrier is a mono- and/or multi-filament metal barrier.

20. The intravaginal ring device according to claims 14-19, wherein the metal and/or polymeric portion of the barrier comprises one or more spermiostatic metals and/or metal salts as active ingredients.

21. The intravaginal ring device according to claim 20, wherein the metal is copper and/or silver, and the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

22. The intravaginal ring device according to claim 1, wherein the barrier is a perforated film.

23. The intravaginal ring device of claim 23 wherein the perforated film comprises multiple layers that are offset.

24. The intravaginal ring device of any one of claim 22 or 23, wherein the perforated film is made with one or more polymers selected from 1,2-polybutadiene, ethylene vinyl acetate, polyethylene, silicone gel, or polyurethane.

25. The intravaginal ring device of any one of claims 22-24, wherein the perforated film comprises one or more spermiostatic metals and/or metal salts as active ingredients.

26. The intravaginal ring device according to claim 25, wherein the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

27. The intravaginal ring device according to any one of claims 1-26, wherein the ring further comprises ascorbic acid.

28. The intravaginal ring device according to any one of claims 1-27, further comprising a removal tab, tag or string.

29. The intravaginal ring device according to any one of claims 1-28, wherein the ring contains two or more segments.

30. The intravaginal ring of any one of claims 1-29, wherein the ring further comprises an active agent to prevent pregnancy, and/or to treat or prevent bacterial infections, and viral infections.

31. The intravaginal ring of claim 30 wherein the active agent is a non-hormonal or hormonal contraceptive, and the antiviral agent is used to treat or prevent one or more sexually transmitted diseases.

32. The intravaginal ring of claim 31, wherein the antiviral agent is one or more of tenofovir, atanzanavir, darunavir, fos/amprenavir, indinavir, lopinavir, nelfinavir, ritonavir, saquinavir, tipranavir, efavirenz, or nelfinavir;

33. The intravaginal ring of claim 31, wherein the hormonal contraceptives is one or more of desogestrel, drospirenone, ethinyl estradiol, levonorgestrel, medroxyprogesterone acetate, norelgestromin, norethindrone, norgestimate, or norgestrel.

34. The intravaginal ring of any one of 30-33, wherein the intravaginal ring further comprises active ingredients contained in discrete reservoirs and/or continuous sheathed in a polymer as part of the uncoated thermoplastic elastomer.

35. The intravaginal ring device according to any one of claims 1-34 wherein the intravaginal ring has less distortion after storage or after insertion into a subject for at least 14 days when compared to an intravaginal ring device made with a copolymeric silicone matrix in which both rings contain equivalent amounts and composition of nonhormonal spermiostatic agents and/or ascorbic acid if present.

36. An intravaginal ring device comprising a saddle-shaped, oval ring made with a non-segmented or segmented uncoated thermoplastic elastomer, excluding copolymeric silicone, wherein the ring contains one or more active ingredients; and, wherein the ring encircles a curved, non-resorbable metal barrier, polymeric barrier, combination metal and polymer barrier or a barrier made with perforated film, wherein the barrier is attached to the ring and the barrier is not fully occlusive to allow the passage of uterine secretions.

37. The intravaginal ring device according to claim 36, wherein the thermoplastic elastomer is selected from one or more of styrene-butadiene block copolymer, ethylene vinyl acetate copolymer, poly(methyl methacrylate), poly(butyl methacrylate), poly(vinylchloride), nylon, soft nylon, poly(ethylene terephthalate) (PET), poly(ethylene), poly(acrylonitrile), PCTFE, poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

38. The intravaginal device according to claim 37, wherein the thermoplastic elastomer is selected from ethylene vinyl acetate copolymer, polyurethane, or PET.

39. The intravaginal ring device according to claim 38, wherein the uncoated thermoplastic elastomer is ethylene vinyl acetate copolymer.

40. The intravaginal ring device according to any one of claims 36-39, wherein the polymeric barrier is a mesh comprised of one or more polyolefin, nylon, and/or silk.

41. The intravaginal ring device of claim 40, wherein the polyolefin is polypropylene or polyethylene.

42. The intravaginal ring device according to any one of claims 36-41, wherein the polymeric mesh is a mono- or multi-filament polymer.

43. The intravaginal ring device according to claim 42, wherein the ring contains one or more spermiostatic metals and/or metal salts as active agents.

44. The intravaginal ring device according to claim 43, wherein the spermiostatic metals and or metal salts are selected from one or more of calcium chloride, magnesium chloride, ferrous sulfate, ferrous gluconate, iron amino acid chelates, copper sulfate, copper gluconate, silver nitrite, copper amino acid chelates, and copper oxide.

45. The intravaginal ring device according to any one of claims 36-39, wherein the barrier is metal and comprises one or more of aluminum, copper, stainless steel, titanium, nickel, nickel-titanium, gold, silver, platinum or palladium.

46. The intravaginal ring device according to claim 45, wherein the metal barrier is a mono- and/or multi-filament.

47. The intravaginal ring device according to claim 46, wherein the metal barrier comprises one or more spermiostatic metals as active agents.

48. The intravaginal ring device according to claim 47, wherein the spermiostatic metal is copper and/or silver.

49. The intravaginal ring device according to any one of claims 36-39, wherein the barrier is a combination of one or more metals and one or more polymers wherein the barrier is attached to the ring.

50. The intravaginal ring device according to claim 49, wherein the polymeric barrier portion of the barrier is a mesh comprised of one or more polyolefin, nylon, and/or silk.

51. The intravaginal ring device of claim 50, wherein the polyolefin is polypropylene or polyethylene.

52. The intravaginal ring device according to any one of claim 50 or 51, wherein the polymeric mesh is a mono- or multi-filament polymer.

53. The intravaginal ring device according to any one of claims 49-52, wherein the metal portion of the barrier comprises a metal selected from one or more of aluminum, copper, stainless steel, titanium, nickel, nickel-titanium, gold, silver, platinum or palladium.

54. The intravaginal ring device according to claim 53, wherein the metal barrier is a mono- and/or multi-filament.

55. The intravaginal ring device according to claims 49-54, wherein the metal and/or polymeric portion of the barrier comprises one or more spermiostatic metals or and/or metal salts as active ingredients.

56. The intravaginal ring device according to claim 55, wherein the metal is copper and/or silver and the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

57. The intravaginal ring device according to claim 36, wherein the barrier is a perforated film.

58. The intravaginal ring device of claim 57 wherein the perforated film comprises multiple layers that are offset.

59. The intravaginal ring device of any one of claim 57 or 58, wherein the perforated film is made with one or more polymers selected from 1,2-polybutadiene, ethylene vinyl acetate, polyethylene, silicone gel, or polyurethane.

60. The intravaginal ring device of any one of claim 59, wherein the perforated film comprises one or more spermiostatic metal or metal salts as active ingredients.

61. The intravaginal ring device according to claim 60, wherein the metal is copper and/or silver and the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

62. The intravaginal ring device according to any one of claims 36-56, wherein the intravaginal ring device further comprises ascorbic acid.

63. The intravaginal ring device according to any one of claims 36-62, further comprising a removal tab, tag or string.

64. The intravaginal ring device according to any one of claims 36-63, wherein the ring contains two or more segments.

65. The intravaginal ring device of any one of claims 36-64, further comprising an active agent in the ring portion to prevent pregnancy, and/or to treat or prevent bacterial infections, or viral infections.

66. The intravaginal ring of claim 59 wherein the active agent to prevent pregnancy is a hormonal contraceptive, and the antiviral agent prevents one or more sexually transmitted infections.

67. The intravaginal ring of any one of claim 65 or 66, wherein the antiviral agent is one or more of tenofovir, atanzanavir, darunavir, fos/amprenavir, indinavir, lopinavir, nelfinavir, ritonavir, saquinavir, tipranavir, efavirenz, or nelfinavir;

68. The intravaginal ring of any one of claim 65 or 66, wherein the hormonal contraceptives is one or more of desogestrel, drospirenone, ethinyl estradiol, levonorgestrel, medroxyprogesterone acetate, norelgestromin, norethindrone, norgestimate, or norgestrel.

69. The intravaginal ring of any one of 65-68, wherein the intravaginal ring further comprises active ingredients contained in discrete reservoirs and/or continuous sheathed in a polymer as part of the thermoplastic elastomer.

70. The intravaginal ring device according to any one of 34-69 wherein the intravaginal ring has less distortion after storage or after insertion into a subject for at least 14 days when compared to an intravaginal ring device made with a copolymeric silicone matrix in which both rings contain equivalent amounts and composition of nonhormonal spermiostatic agents and/or ascorbic acid if present.

71. An intravaginal ring device comprising a flat, circular or oval ring made with a non-segmented or segmented uncoated thermoplastic elastomer, excluding copolymeric silicone, wherein the ring encircles a curved or flat, non-resorbable metal barrier, polymeric barrier, combination metal and polymer barrier or a barrier made with perforated film, wherein the barrier is attached to the ring and said metal and polymer barriers and said perforated film contain active ingredients; wherein said barriers or perforated film is not fully occlusive to allow the passage of uterine secretions.

72. The intravaginal ring device according to claim 71, wherein the uncoated thermoplastic elastomer is selected from one or more of styrene-butadiene block copolymer, ethylene vinyl acetate copolymer, poly(methyl methacrylate), poly(butyl methacrylate), poly(vinylchloride), nylon, plasticized soft nylon, poly(ethylene terephthalate) (PET), poly(ethylene), poly(acrylonitrile), polychlorotrifluoroethylene (PCTFE), poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

73. The intravaginal device according to claim 72, wherein the uncoated thermoplastic elastomer is selected from ethylene vinyl acetate copolymer, polyurethane, or PET.

74. The intravaginal ring device according to claim 73, wherein the uncoated thermoplastic elastomer is ethylene vinyl acetate copolymer.

75. The intravaginal ring device according to any one of claims 71-74, wherein the polymeric barrier is a mesh comprised of one or more of polyolefin, nylon, and/or silk.

76. The intravaginal ring device of claim 75, wherein the polyolefin is polypropylene or polyethylene.

77. The intravaginal ring device according to any one of claims 71-76 wherein the polymeric mesh is a mono- or multi-filament polymer.

78. The intravaginal ring device according to claim 77, wherein the polymeric barrier or mesh comprises one or more metal salts as active ingredients.

79. The intravaginal ring device according to claim 78, wherein the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

80. The intravaginal ring device according to any one of claims 71-74, wherein the barrier is metal and the barrier comprises one or more of aluminum, copper, stainless steel, titanium, nickel, nickel-titanium, gold, silver, platinum or palladium.

81. The intravaginal ring device according to 80, wherein the metal barrier is a mono- and/or multi-filament metal barrier.

82. The intravaginal ring device according to claim 81, wherein the metal barrier comprises one or more spermiostatic metals as active ingredients.

83. The intravaginal ring device according to claim 82, wherein the metal is copper and/or silver.

84. The intravaginal ring device according to any one of claims 71-74, wherein the barrier is a combination of metal and a polymer barrier which is attached to the ring.

85. The intravaginal ring device according to claim 84, wherein the polymeric barrier portion is a mesh comprised of one or more polyolefin, nylon, and/or silk.

86. The intravaginal ring device of claim 85, wherein the polyolefin is polypropylene or polyethylene.

87. The intravaginal ring device according to any one of claim 85 or 86, wherein the polymeric mesh is a mono- or multi-filament polymer.

88. The intravaginal ring device according to any one of claims 84-88, wherein the metal portion of the barrier comprises one or more of aluminum, copper, stainless steel, titanium, nickel, nickel-titanium, gold, silver, platinum or palladium.

89. The intravaginal ring device according to claim 88, wherein the metal barrier is a mono- or multi-filament polymer.

90. The intravaginal ring device according to claims 84-89, wherein the metal and/or polymeric portion of the barrier comprises one or more spermiostatic metals and/or metal salts as active ingredients.

91. The intravaginal ring device according to claim 90, wherein the metal is copper and/or silver and the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

92. The intravaginal ring device according to claim 71-74, wherein the barrier is a perforated film.

93. The intravaginal ring device of claim 92 wherein the perforated film comprises multiple layers that are offset.

94. The intravaginal ring device of any one of claim 92 or 93, wherein the perforated film is made with polymers selected from 1,2-polybutadiene, ethylene vinyl acetate, polyethylene, silicone gel, or polyurethane.

95. The intravaginal ring device of any one of claims 92-94, wherein the perforated film comprises one or more spermiostatic metals and/or metal salts as active ingredients.

96. The intravaginal ring device according to claim 95, wherein the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

97. The intravaginal ring device according to any one of claims 71-96, wherein the intravaginal ring further comprises ascorbic acid.

98. The intravaginal ring device according to any one of claims 71-97, further comprising a removal tab, tag or string.

99. The intravaginal ring device according to any one of claims 71-98, wherein the ring contains two or more segments.

100. The intravaginal ring device according to any one of 71-99 wherein the intravaginal ring has less distortion after storage or after insertion into a subject for at least 14 days when compared to an intravaginal ring device made with a copolymeric silicone matrix in which both rings contain equivalent amounts and composition of nonhormonal spermiostatic agents and/or ascorbic acid if present.

101. An intravaginal ring device comprising a saddle-shaped, oval ring made with a non-segmented or segmented uncoated thermoplastic elastomer, excluding copolymeric silicone, wherein the ring encircles a curved or flat, non-resorbable metal barrier, polymeric barrier, combination metal and polymer barrier or a barrier made with perforated film, wherein the barrier is attached to the ring and said metal and polymer barriers and said perforated film optionally contain active ingredients; wherein said barriers or perforated film is not fully occlusive to allow the passage of uterine secretions.

102. The intravaginal ring device according to claim 101, wherein the thermoplastic elastomer is selected from one or more of styrene-butadiene block copolymer, ethylene vinyl acetate copolymer, poly(methyl methacrylate), poly(butyl methacrylate), poly(vinylchloride), nylon, soft nylon, poly(ethylene terephthalate) (PET), poly(ethylene), poly(acrylonitrile), PCTFE, poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

103. The intravaginal device according to claim 102, wherein the uncoated thermoplastic elastomer is selected from ethylene vinyl acetate copolymer, polyurethane, or PET.

104. The intravaginal ring device according to claim 103, wherein the uncoated thermoplastic elastomer is ethylene vinyl acetate copolymer.

105. The intravaginal ring device according to claim any one of claims 101-104, wherein the polymeric barrier is a mesh comprised of one or more polyolefin, nylon, and/or silk.

106. The intravaginal ring device of claim 105, wherein the polyolefin is polypropylene or polyethylene.

107. The intravaginal ring device according to any one of claims 101-106, wherein the polymeric mesh is a mono- or multi-filament polymer.

108. The intravaginal ring device according to claim 107, wherein the polymeric mesh contains one or more spermiostatic metal salts as active ingredients.

109. The intravaginal ring device according to claim 99, wherein the spermiostatic metal salts are selected from one or more of calcium chloride, magnesium chloride, ferrous sulfate, ferrous gluconate, iron amino acid chelates, copper sulfate, copper gluconate, silver nitrite, copper amino acid chelates, and copper oxide.

110. The intravaginal ring device according to any one of claims 101-104, wherein the barrier is metal comprising one or more of aluminum, copper, stainless steel, titanium, nickel, nickel-titanium, gold, silver, platinum or palladium.

111. The intravaginal ring device according to claim 110, wherein the metal barrier is a mono- and/or multi-filament.

112. The intravaginal ring device according to claim 111, wherein the metal barrier comprises one or more spermiostatic metals as active ingredients.

113. The intravaginal ring device according to claim 112, wherein the metal is copper and/or silver.

114. The intravaginal ring device according to any one of claims 101-104, wherein the barrier is a combination of one or more metals and one or more polymers wherein the barrier is attached to the ring.

115. The intravaginal ring device according to claim 114, wherein the polymeric barrier portion of the barrier is a mesh comprised of one or more polyolefin, nylon, and/or silk.

116. The intravaginal ring device of claim 115, wherein the polyolefin is polypropylene or polyethylene.

117. The intravaginal ring device according to any one of claims 115-116, wherein the polymeric mesh is a mono- or multi-filament polymer.

118. The intravaginal ring device according to any one of claims 114-117, wherein the metal portion of the barrier comprises a metal selected from one or more of aluminum, copper, stainless steel, titanium, nickel, nickel-titanium, gold, silver, platinum or palladium.

119. The intravaginal ring device according to claim 118, wherein the metal barrier is a mono- and/or multi-filament.

120. The intravaginal ring device according to claims 114-119, wherein the metal and/or polymeric portion of the barrier comprises one or more spermiostatic metals and/or metal salts as active ingredients.

121. The intravaginal ring device according to claim 110, wherein the metal is copper and/silver and the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

122. The intravaginal ring device according to claim 101-104, wherein the barrier is a perforated film.

123. The intravaginal ring device of claim 122 wherein the perforated film comprises multiple layers that are offset.

124. The intravaginal ring device of any one of claim 122 or 123, wherein the perforated film is made with polymers selected from 1,2-polybutadiene, ethylene vinyl acetate, polyethylene, silicone gel, or polyurethane.

125. The intravaginal ring device of any one of claims 122-124, wherein the perforated film comprises one or more spermiostatic metals and/or metal salts as active ingredients.

126. The intravaginal ring device according to claim 125, wherein the metal salt is selected from one or more of calcium chloride, magnesium chloride, ferrous sulfate, ferrous gluconate, iron amino acid chelates, copper sulfate, copper gluconate, silver nitrite, copper amino acid chelates, and copper oxide.

127. The intravaginal ring device according to any one of claims 101-126, wherein the intravaginal ring further comprises ascorbic acid.

128. The intravaginal ring device according to any one of claims 101-127, further comprising a removal tab, tag or string.

129. The intravaginal ring device according to any one of claims 101-128, wherein the ring contains two or more segments.

130. The intravaginal ring device according to any one of 101-129 wherein the intravaginal ring has less distortion after storage or after insertion into a subject for at least 14 days when compared to an intravaginal ring device made with a copolymeric silicone matrix in which both rings contain equivalent amounts and composition of nonhormonal spermiostatic agents and/or ascorbic acid if present.

131. An intravaginal ring device comprising a flat, circular or oval ring made with a non-segmented uncoated thermoplastic elastomer, excluding copolymeric silicone, wherein the ring contains one or more non-hormonal spermiostatic agents; and, wherein the ring encircles a curved, non-resorbable polymeric barrier which is attached to the ring; and wherein the ring covers the cervix when inserted into a subject.

132. The intravaginal ring device according to claim 131, wherein the uncoated thermoplastic elastomer is selected from one or more of styrene-butadiene block copolymer, ethylene vinyl acetate copolymer, poly(methyl methacrylate), poly(butyl methacrylate), poly(vinylchloride), nylon, plasticized soft nylon, poly(ethylene terephthalate) (PET), poly(ethylene), poly(acrylonitrile), polychlorotrifluoroethylene (PCTFE), poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

133. The intravaginal device according to claim 132, wherein the uncoated thermoplastic elastomer is selected from ethylene vinyl acetate copolymer, polyurethane, or PET.

134. The intravaginal ring device according to claim 133, wherein the uncoated thermoplastic elastomer is ethylene vinyl acetate copolymer.

135. The intravaginal ring device according to any one of claims 131-134, wherein the polymeric barrier is a mesh comprised of one or more polyolefin, nylon, and/or silk.

136. The intravaginal ring device of claim 135, wherein the polyolefin is polypropylene or polyethylene.

137. The intravaginal ring device according to any one of claims 131-136 wherein the mesh or barrier is a mono- or multi-filament polymer.

138. The intravaginal ring device according to any one of claims 131-137, wherein the ring contains one or more spermiostatic metals and/or metal salts.

139. The intravaginal ring device according to claim 138, wherein the ring contains spermiostatic metals and/or salts selected from one or more of ferrous sulfate, ferrous gluconate, iron amino acid chelates, copper sulfate, copper gluconate, copper amino acid chelates, and copper oxide.

140. The intravaginal ring device according to claim 139, wherein the ring further comprises ascorbic acid.

141. The intravaginal ring device according to claim 140, further comprising a removal tab.

142. The intravaginal ring device according to any one of claims 131-141 wherein the intravaginal ring has less distortion after storage or after insertion into a subject for at least 14 days when compared to an intravaginal ring device made with a copolymeric silicone matrix in which both rings contain equivalent amounts and composition of nonhormonal spermiostatic agents and/or ascorbic acid if present.

143. An intravaginal ring device comprising a saddle-shaped, oval ring made with a non-segmented uncoated thermoplastic elastomer, excluding copolymeric silicone, wherein the ring contains one or more non-hormonal spermiostatic agents; and, wherein the ring encircles a curved, non-resorbable polymeric barrier which is attached to the ring; and wherein the ring covers the cervix.

144. The intravaginal ring device according to claim 143, wherein the thermoplastic elastomer is selected from one or more of styrene-butadiene block copolymer, ethylene vinyl acetate copolymer, poly(methyl methacrylate), poly(butyl methacrylate), poly(vinylchloride), nylon, soft nylon, poly(ethylene terephthalate) (PET), poly(ethylene), poly(acrylonitrile), PCTFE, poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), polypropylene or other poly(olefins).

145. The intravaginal device according to claim 144, wherein the thermoplastic elastomer is selected from ethylene vinyl acetate copolymer, polyurethane, or PET.

146. The intravaginal ring device according to claim 145, wherein the uncoated thermoplastic elastomer is ethylene vinyl acetate copolymer.

147. The intravaginal ring device according to claim any one of claims 143-146, wherein the barrier is a mesh comprised of one or more polyolefin, nylon, and/or silk.

148. The intravaginal ring device of claim 147, wherein the polyolefin is polypropylene or polyethylene.

149. The intravaginal ring device according to claims any one of claims 143-148, wherein the mesh and/or barrier is a mono- or multi-filament polymer.

150. The intravaginal ring device according to any one of claims 143-149, wherein the ring contains one or more spermiostatic metals and/or metal salts.

151. The intravaginal ring device according to claim 150, wherein the spermiostatic metals and or metal salts are selected from one or more of ferrous sulfate, ferrous gluconate, iron amino acid chelates, copper sulfate, copper gluconate, copper amino acid chelates, copper oxide.

152. The intravaginal ring device according to claims any one of 143-151 wherein the intravaginal ring has less distortion after storage or after insertion into a subject for at least 14 days when compared to an intravaginal ring device made with a copolymeric silicone matrix in which both rings contain equivalent amounts and composition of nonhormonal spermiostatic agents and/or ascorbic acid if present.

153. An intravaginal ring device comprising a flat, circular or oval ring made with a segmented uncoated thermoplastic elastomer, excluding copolymeric silicone, with a removal tab, wherein the ring contains one or more non-hormonal spermiostatic agents; and, wherein the ring encircles a curved, non-resorbable polymeric barrier which is attached to the ring; and wherein the ring covers the cervix when inserted into a subject.

154. The intravaginal ring device according to claim 153, wherein the uncoated thermoplastic elastomer is selected from one or more of styrene-butadiene block copolymer, ethylene vinyl acetate copolymer, poly(methyl methacrylate), poly(butyl methacrylate), poly(vinylchloride), nylon, soft nylon, poly(ethylene terephthalate) (PET), poly(ethylene), poly(acrylonitrile), PCTFE, poly(ethylene-vinyl esters), poly(ethylene-vinyl acetate), poly(vinylchloride-diethyl fumarate), poly(esters of acrylic and methacrylic), poly(amides), poly(vinyl chloride), PTFE (polytetrafluoroethylene) poly(urethane), or polypropylene or other poly(olefins).

155. The intravaginal ring device according to claim 154, wherein the thermoplastic elastomer is selected from ethylene vinyl acetate copolymer, polyurethane, or PET.

156. The intravaginal ring device according to claim 155, wherein the thermoplastic elastomer is ethylene vinyl acetate copolymer.

157. The intravaginal ring device according to any one of claims 153-156, wherein the barrier is a mesh comprised of one or more polyolefin, nylon, and/or silk.

158. The intravaginal ring device of claim 157, wherein the polyolefin is polypropylene or polyethylene.

159. The intravaginal ring device according to any one of claims 153-158 wherein the barrier or mesh is a mono- or multi-filament polymer.

160. The intravaginal ring device according to any one of claims 153-159, wherein the intravaginal ring device contains one or more spermiostatic metals and/or metal salts.

161. The intravaginal ring device according to any one of claims 153-160, wherein the spermiostatic metals and or salts are selected from one or more of ferrous sulfate, ferrous gluconate, iron amino acid chelates, copper sulfate, copper gluconate, copper amino acid chelates, copper oxide.

162. The intravaginal ring device according to claim 161, wherein one segment of the ring contains spermiostatic metals and or metal salts and another segment further comprises ascorbic acid.

163. The intravaginal device according to any one of claims 153-162, wherein the intravaginal ring has less distortion after storage or after insertion into a subject for at least a week when as compared to an intravaginal ring device with at least two segments made with a copolymeric silicone matrix in which both rings contain equivalent amounts and composition of nonhormonal spermiostatic agents and ascorbic acid.

164. The intravaginal ring device according to any one of claims 153-162 wherein the intravaginal ring has less distortion after storage in packaging containing or after insertion into a subject for at least 14 days when compared to an intravaginal ring device made with a copolymeric silicone matrix in which both rings contain equivalent amounts and composition of nonhormonal spermiostatic agents and/or ascorbic acid if present.
